(19) United States
(12) Patent Application Publication
(10) Pub. No.: US 2026/0207815 A1
(43) Pub. Date: Jul. 23, 2026

(54) CELL-BINDING MOTIFS (CBMs) FUNCTIONALIZED POLYMERS

(71) Applicants: UNIVERSITEIT GENT, Gent (BE); VRIJE UNIVERSITEIT BRUSSEL, Brussel (BE)

(72) Inventors: Quinten THIJSSEN, Gent (BE); Sandra VAN VLIERBERGHE, Gent (BE); Kevin VAN HOLSBEECK, Brussel (BE); Steven BALLET, Brussel (BE)

(21) Appl. No.: 19/138,861

(22) PCT Filed: Dec. 12, 2023
(86) PCT No.: PCT/EP2023/085429

§ 371 Date: Jun. 13, 2025

(30) Foreign Application Priority Data
Dec. 13, 2022 (EP) 22213201.1

(51) Int. Cl. A61L 27/18; A61L 27/58; C08L 67/04

(52) U.S. Cl. CPC A61L 27/18; A61L 27/58; C08L 67/04; A61L 2300/25

(57) ABSTRACT

The present invention pertains to the field of tissue engineering and in particular to materials adapted to adhere to cells. More in particular, the present invention pertains to polymer comprising thiol-ene crosslinked networks exposing one or more cell-binding-motifs (CBMs) and combinations providing for said networks.

### FIELD OF THE INVENTION

The present invention pertains to the field of tissue engineering and in particular materials adapted to adhere to cells. More in particular, the present invention pertains to polymers, such as polyesters comprising thiol-ene crosslinked networks exposing one or more cell-binding-motifs and combinations providing for said networks.

### BACKGROUND TO THE INVENTION

The interactions between cells and materials are manyfold and can be considered highly complex. Among those interactions, cell adhesion has been of great interest in biomedical research. It can be considered as a sequential process of attachment, spreading and growth of the involved cells. Moreover, it is crucial for further cell migration, differentiation and the production of extra cellular matrix (ECM) components, illustrating its importance. Many natural materials have this innate ability to induce cell adhesion of which the most well-known are fibronectin and gelatin. These materials contain cell-binding-motifs (CBMs) that are able to induce cell adhesion. Most CBMs are short peptide sequences such as the tripeptide Arg-Gly-Asp (i.e. RGD). Advances in peptidomimetics and peptide synthesis have paved the way towards synthetic CBMs that can be modified to include certain linkers or binding sites (i.e. cysteine). However, in contrast to natural materials, synthetic materials such as poly(ε-caprolactone) (PCL) are considered non-cell-adhesive. Nevertheless, these synthetic materials can be considered the broadest and most divergent class of materials exploited in the context of tissue engineering due to their mechanical stability, processability, degradability and tunability.

In an attempt to mimic the innate cell-adhesive abilities of nature (i.e. gelatin), synthetic RGD has been grafted onto the surface of PCL. Multiple groups have successfully shown that by doing so, cell-adhesion can be introduced onto the surface of synthetic polymers such as PCL. However, this surface functionalization of PCL includes multiple steps and is often un-controlled and mediated by harsh conditions. More precisely, first an excess of di-amine is employed to introduce a linker via a nucleophilic substitution at the ester bond on the PCL surface. Subsequently, a di-epoxide or di-aldehyde is coupled onto this linker. Finally, RGD is coupled onto the residual functionality of the linker via its N-terminal amine moiety.

Causa et al., 2010, discloses a method for the immobilization of CBMs on the surface of a polyester (PCL). In particular, Causa et al., 2010, discloses the functionalization of PCL polymer by a two-step process consisting of (1) aminolysis to graft functional groups (primary amines) on the film surface and a following (2) conjugation of the RGD motif.

Zhang et al., 2009, have reported coupling RGD onto PCL via an additional cysteine moiety and a maleimide activated linker or via EDC/NHS coupling chemistry. It should be noted that these modifications are limited to the surface of the material and that the harsh conditions imply local degradation of the PCL matrix. Additionally, the cell-adhesive properties can be expected to disappear rapidly when cells penetrate into the material or when the surface degrades over time.

Here is hence the need for polymeric networks having cell adhesion properties wherein the CBMs are provided both at the surface and in the bulk of the network, polymers and combination providing for this.

### SUMMARY OF THE INVENTION

In a first aspect, the present invention relates to a combination comprising:
a polymer, preferably a degradable synthetic polymer, more preferably a non-cell-adhesive (bio) degradable synthetic polymer, such as polyesters, such as poly(ε-caprolactone) (PCL), poly(lactic acid) (PLA), poly(lactic-co-glycolic acid) (PLGA), polyglycolic acid (PGA); poly(ortho-esters), and polycarbonates, such as poly(trimethylene) carbonate, and copolymers thereof, the polymer having a first thiol-ene crosslinkable group, being either a thiol comprising group, such as a C-terminal cysteine residue, or a -ene comprising group;
a CBM comprising compound of formula (I):

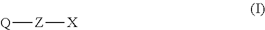

wherein:
   Q is a group comprising a cell-binding motif (CBM), such as an RGD motif;
   Z is a spacer, optionally present, and preferably from 0 to 30 atoms in length preferably from 0 to 10 atoms in length;
   X is a group comprising a second thiol-ene crosslinkable group; and
wherein at least one of said first and second thiol-ene crosslinkable group is a thiol comprising group and at least one of said first and second thiol-ene crosslinkable group is a -ene comprising group.

Specifically, the present invention relates to a combination comprising:
a polymer, preferably a degradable synthetic polymer, having a first thiol-ene crosslinkable group;
a CBM comprising compound of formula (I):

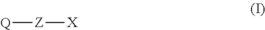

wherein:
   Q is a group comprising a CBM;
   Z is a spacer, optionally present;
   X is a group comprising a second thiol-ene crosslinkable group
wherein at least one of said first and second thiol-ene crosslinkable group is a thiol comprising group and at least one of said first and second thiol-ene crosslinkable group is a -ene comprising group;
wherein the polymer is selected from: polyesters, polyorthoesters, polycarbonates, and copolymers thereof; and
wherein the polymer is star-shaped.

According to an embodiment of the present invention, the polymer is a polyester selected from: poly(ε-caprolactone) (PCL), poly(lactic acid) (PLA), poly(lactic-co-glycolic acid) (PLGA), and polyglycolic acid (PGA).

According to a further embodiment of the present invention, the spacer Z is from 0 to 30 atoms in length.

According to yet a further embodiment of the present invention, the spacer Z is from 0 to 10 atoms in length.

According to yet a further embodiment of the present invention, the spacer Z is from 1 to 30 atoms in length, preferably from 1 to 10 atoms in length.

According to yet a further embodiment of the present invention, the spacer Z is of formula (II):

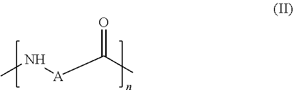

wherein:
   A is a bivalent radical, optionally comprising one or more heteroatoms; and
   n≥1, preferably 1 $ n $4. According to yet a further embodiment of the present invention, n is 1.

According to a particular embodiment of the present invention, n is 1.

According to yet a further embodiment of the present invention, the CBM comprises an RGD motif.

According to yet a further embodiment of the present invention, the first thiol-ene crosslinkable group is a C—C double bond comprising group and the second thiol-ene crosslinkable group is a —SH comprising group.

According to yet a further embodiment of the present invention, wherein the second thiol-ene crosslinkable group is a C-terminal cysteine residue.

According to yet a further embodiment of the present invention, the polymer has an alkene content from 0.0001 mol/g to 0.01 mol/g, preferably from 0.005 mol/g to 0.01 mol/g.

According to yet a further embodiment of the present invention, the polymer has a molar mass from 500 g/mol to 20000 g/mol.

According to a further aspect, the present invention pertains to a polymer network, such as a polyester polymer network, comprising an outer or surface region and an inner or bulk region, said surface region and said bulk region comprising cell-binding motifs (CBMs).

Specifically, the present invention relates to a polymer network comprising:
an outer region;
an inner region; and
a plurality of cell-binding motifs (CBMs), provided within the outer region and the inner region.

According to a specific embodiment, the present invention provides a polymer network in a crosslinked state.

According to a more specific embodiment, the present invention provides a polymer network, comprising the combination as defined herein, in a crosslinked state.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1, also referred to as FIG. 1, illustrates the structure of the considered RGD-containing peptides bearing a C-terminal cysteine residue.

FIG. 2, also referred to as FIG. 2, illustrates the synthesis of three-arm PCL via metathesis ring opening polymerization and subsequent functionalization towards alkene-functionalized PCL using allyl isocyanate.

FIG. 3, also referred to as FIG. 3, illustrates the schematic depiction of the RGD-functionalized PCL via thiol-ene photo-crosslinking upon UVA-irradiation (365 nm, 10 mW/cm²) in presence of TPO-L as photo-initiator.

FIG. 4, also referred to as FIG. 4, illustrates A. Gel fraction; B. swelling degree of the RGD-functionalized PCLs.

FIG. 5, also referred to as FIG. 5, illustrates the static contact angle measurements (SCA) of the RGD-functionalized PCLs to evaluate the influence of the modification on hydrophilicity of the materials surfaces.

FIG. 6, also referred to as FIG. 6, illustrates A. Graph illustrating the amount of cells that are alive per μm² determined via live/dead assay of HFFs after culturing for 1, 3 and 7 days on the RGD-functionalized PCLs; B. Metabolic activity determined via MTS assay of HFFs after 1 day of culturing on the RGD-functionalized PCLs. Experiments were performed in triplicate; C visualization of living human foreskin fibroblasts (HFFs), after 7 days, cultured on the different RGD-functionalized PCLs.

FIG. 7, also referred to as FIG. 7, illustrates the quantification of living adipose derived stem cells (ADSCs), after 1, 3 and 7 days, on the surface of 3D photo-crosslinked polyester scaffolds, obtained by volumetric 3D-printing of a photoresist that contained RGD as cell-binding motif.

FIG. 8, also referred to as FIG. 8, illustrates the visualization of living adipose derived stem cells (ADSCs), after 7 days, on the surface of 3D photo-crosslinked polyester scaffolds, obtained by volumetric 3D-printing of a photoresist that contained RGD as cell-binding motif.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. When describing the compounds of the invention, the terms used are to be construed in accordance with the following definitions, unless a context dictates otherwise.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed. Further, by means of the wording a, reference is made to one or more polyesters.

The term “alkyl” by itself or as part of another substituent refers to a fully saturated hydrocarbon of Formula CₓH₂ₓ₊₁ wherein x is a number greater than or equal to 1. Generally, alkyl groups of this invention comprise from 1 to 20 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl, and its isomers (e.g., n-butyl, i-butyl and t-butyl); pentyl and its isomers, hexyl and its isomers, heptyl and its isomers, octyl and its isomers, nonyl and its isomers; decyl and its isomers. C₁-C₆ alkyl includes all linear, branched, or cyclic alkyl groups with between 1 and 6 carbon atoms, and thus includes methyl, ethyl, n-propyl, i-propyl, butyl and its isomers (e.g., n-butyl, i-butyl and t-butyl); pentyl and its isomers, hexyl and its isomers, cyclopentyl, 2-, 3-, or 4-methylcyclopentyl, cyclopentylmethylene, and cyclohexyl. The term “heterocyclic” as used herein by itself or as part of another group refer to non-aromatic, fully saturated or partially unsaturated cyclic groups (for example, 3 to 13 member monocyclic, 7 to 17 member bicyclic, or 10 to 20 member tricyclic ring systems, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized, and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms.

Exemplary heterocyclic groups include piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, phthalazinyl, oxetanyl, thietanyl, 3-dioxolanyl, 1,3-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, and morpholinyl. The term “cyclic”, “cyclic alkyl” or “cycloalkyl” as used herein by itself or as part of another substituent is a cyclic alkyl group, that is to say, a monovalent, saturated, or unsaturated hydrocarbyl group having 1, 2, or 3 cyclic structure. Cycloalkyl includes all saturated or partially saturated (containing 1 or 2 double bonds) hydrocarbon groups containing 1 to 3 rings, including monocyclic, bicyclic, or polycyclic alkyl groups. Cycloalkyl groups may comprise 3 or more carbon atoms in the ring and generally, according to this invention comprise from 3 to 15 atoms. The further rings of multi-ring cycloalkyls may be either fused, bridged and/or joined through one or more spiro atoms. Examples of cycloalkyl groups include but are not limited to cyclopropyl, cyclobutyl, cyclopentyl, cyclohexyl, cycloheptyl, cyclooctyl, cyclononyl, adamantanyl and cyclodecyl.

In a first aspect, the present invention relates to a combination comprising
a polymer having a first thiol-ene crosslinkable group;
a CBM comprising compound of formula (I):

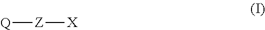

wherein:
   Q is a group comprising a cell-binding motif (CBM);
   Z is a spacer, optionally present;
   X is a group comprising a second thiol-ene crosslinkable group; and
wherein at least one of said first and second thiol-ene crosslinkable group is a thiol comprising group and at least one of said first and second thiol-ene crosslinkable group is a -ene comprising group.

The present invention provides for several advantages. The combination in accordance with the present invention advantageously provides for crosslinked networks comprising CBMs chemically linked throughout the bulk of the crosslinked network. In other words, the CBM is homogeneously distributed throughout the material. The presence of a CBM is beneficial in improving the cell adherence properties of the polymer networks obtainable from the present combination. In particular, chemically anchoring the CBM throughout the thiol-ene crosslinked polymer network of the present invention significantly increased the number of adhered cells.

The combination according to the present invention also has the advantage of providing a way to spatially controlling cell-adhesion. This is possible because the grafting of the CBM itself is spatially controlled. By using thiol-ene crosslinking to graft the CBM comprising compound to the polymer, which allows for light induced crosslinking, the patterning resolution is really high. This allows to control the location where, on the 3D structure of the polymer, the CBM is introduced.

The combination according to the present invention has the advantage of providing crosslinked networks which can be easily tuned so to provide targeted cell adhesion.

The combination according to the present invention allows for the obtainment of networks which can be spatially controlled by means of selecting the appropriate spacer Z type and length. Furthermore, the combination according to the present invention provides for networks having excellent network connectivity.

Another advantage of the present invention is that the thiol-ene crosslinkable groups of the combination provide for reactant specificity and tolerance of other functional groups. Furthermore, the formation of the polymer network occurs with high conversions in virtually any solvent, including aqueous solvent systems, and also at relatively low temperatures, and potentially also in a melt/solid state.

Further, an additional advantage provided by the present invention is that the polymer networks provide a controllable biodegradation time (i.e., controlled release), so that CBM will be released as a function of degradation.

In accordance with the present invention, by means of the term “combination”, reference is made to the product obtained from combining two or more compounds together. In accordance with the present invention, by means of the term “compound”, reference is made to a chemical compound, in other words, a molecule, of any size e.g., macromolecule such as a polymer or a small molecule. In the present case, the two products combined together are said polymer and said compound defined in accordance with the present invention. In accordance with the present invention, the combination comprises a polymer and a compound of formula (I), which both comprise groups capable of thiol-ene crosslinking, so that the polymer and the compound can be covalently linked via a thiol-ene crosslinking reaction. The combination of the present invention can further comprise other components, such as a photoinitiator and/or a chain transfer agent.

The combination according to the present invention hence comprises a polymer and a CBM comprising compound of formula (I). The polymer of the present invention comprises a first thiol-ene crosslinkable group.

In accordance with the present invention, by means of the term “crosslinkable group”, reference is made to a group provided to crosslink, thereby forming a covalent bond with another group it can react with, by means of a chemical reaction. The chemical reaction (thiol-ene reaction) providing for the polymer network of the present invention can be initiated with or without the intervention of another entity, such as UV light, heat, or another compound. Preferably, the thiol-ene reaction is initiated by irradiating the combination according to the present invention. Photoinitiators, thermal initiators and/or a redox initiators can be used to facilitate crosslinking.

In the context of the present invention, the crosslinkable group provided to both the ester and the CBM comprising compound of formula (I) is a thiol-ene crosslinkable group. For the CBM comprising compound of formula (I), the thiol-ene crosslinkable group is comprised within the X group.

By means of the term “thiol-ene crosslinkable group”, reference is made to a group capable to participate in a thiol-ene reaction, also known as alkene hydrothiolation, wherein a C—S bond is formed. In accordance with the present invention, the first and second thiol-ene crosslinkable groups are selected from either a thiol comprising group or an -ene group. The thiol comprising group and the -ene group are provided to react together thereby forming an C—S bond between the polymer and the CBM comprising compound of formula (I). The first thiol-ene crosslinkable group and the second thiol-ene crosslinkable group are adapted to form a thiol-ene crosslink so that at least one of said first and second thiol-ene crosslinkable group is a thiol comprising group and at least one of said first and second thiol-ene crosslinkable group is a -ene comprising group. Preferably, the first and second crosslinkable groups are reacted in the presence of a photoinitiator.

Examples of suitable photoinitiators are Eosin Y, diphenyl(2,4,6-trimethylbenzoyl) phosphine oxide (TPO), Ethyl (2,4,6-trimethylbenzoyl) phenyl phosphinate (TPO-L), Phenylbis(2,4,6-trimethylbenzoyl) phosphine oxide (BAPO), 4,4′-bis(dimethylamino)benzophenone and Irgacure 2959. The selection of the type of photoinitiator used depends the laser wavelength with which the network is crosslinked. Generally, photo-initiators in the UVA-visible light range (such as TPO, TPO-L, BAPO and Eosin Y) are preferred since these lower energy intensive wavelengths are less damaging for the materials. However, the type of initiator will generally not affect the final material properties. Suitable redox and/or thermal initiators are: 4,4′-Azobis(4-cyanovaleric acid), 1,1′-Azobis(cyclohexanecarbonitrile), 2,2′-Azobis(2-methylpropionamidine) dihydrochloride granular, 2,2′-Azobis(2-methylpropionitrile), Luperox®, Dicumyl peroxide, tert-Butyl hydroperoxide, Cumene hydroperoxide, benzoylperoxide.

With respect to the thiol comprising group, by means of the term “thiol comprising group”, reference is made to at least a part of a molecule comprising at least a thiol functional group, in other words an —SH functional group.

It should be clear to the skilled in the art that it derives that the thiol comprising group can either be attached to the CBM comprising compound of formula (I) or the polymer, and that the -ene group (I) can either be attached to the CBM comprising compound of formula (I) or the polymer. It should also be clear to the skilled in the art that when the thiol comprising group is attached to the CBM comprising compound of formula (I), the -ene group is attached to polymer.

In accordance with a preferred embodiment of the present invention in the combination the polymer has a first thiol-ene crosslinkable group being a C—C double bond and the CBM comprising compound of formula (I) has a second thiol-ene crosslinkable group being an —SH comprising group. An advantage of the present embodiment is that it allows avoiding disulfide bond formation on the polymer, which can be better controlled on the CBM comprising compound (e.g. by purification). This provides a maximal linking of CBM comprising compound to the available positions on the polymer and hence benefits polymer network formation.

In accordance with an embodiment of the present invention, the polymer network can comprise a chain transfer agent, shifting the polymerization from purely chain growth to a combination of both step and chain growth polymerization. Various chain transfer agents could be used to carry out the present embodiment, and can be those comprising at least two thiol groups, such as and not limited to: 1,2-Ethanedithiol, 1,3-Propanedithiol, 1,4-Butanedithiol, 2,2′-Thiodiethanethiol, 2,2′-(Ethylenedioxy) diethanethiol, Trimethylolpropane tris(3-mercaptopropionate), Pentaerythritol tetrakis(3-mercaptopropionate) and combinations thereof.

In accordance with the present invention, by means of the term “-ene group”, reference is made to at least a part of a molecule comprising a carbon double bond, in other words, a C—C double bond, in other words a C═C bond. Preferably, the -ene group comprises a terminal C—C double bond. In accordance with an embodiment, the -ene group is of formula (III):

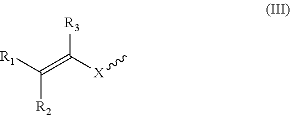

Wherein R₁, R₂, R₃ are independently selected from: H, alkyl, O, N, halogen, S or at least one of R₁, R₂, R₃ together with the group at position X form a cyclic or heterocyclic structure, and X is a group selected from: alkyl, O, N, S, (C═O) N, (C═O) alkyl, O-alkyl, N-alkyl, S-alkyl, —(C═O)—O—.

The -ene group in accordance with the present invention can be part of a variety of functional groups able of providing thiol-ene crosslinking according to the state of the art, such as: allyl, Norbornene, vinyl ether, propenyl, alkene, vinyl ester, N-vinyl amide, allyl ether, allyl triazine, allylisocyanurate, N-substituted maleimide, styrene, conjugated diene.

Various polymers can be used to carry out the present invention and the selection of said polymer is determined by the application in which the polymer networks of the invention are to be used.

The type of polymer used in the combination is dependent on the application. For example, PCL is less brittle and degrades slower (several years), so would be the most suitable for long-term implants whereas PLA is harder and more brittle and degrades relatively fast (several months) and would then be more suitable for drug release/short term implants for example. A difference between polyesters and polycarbonates is that polyesters degrade hydrolytically, while polycarbonates degrade enzymatically.

In summary, depending on the specific application, different biodegradation time would be required, and possibly different mechanical properties. This aforementioned need would determine which type of polymer, e.g. polyester, one would choose. These polymers are all not cell-interactive. Hence, they all require the CBM to make it cell-interactive. In the context of tissue engineering, cell-interactive properties are generally needed to ensure cell adhesion.

The polymer in the combination of the present invention is preferably a degradable synthetic polymer, more preferably a non-cell-adhesive (bio) degradable synthetic polymer. Preferably, the polymer according to the present invention is a biodegradable synthetic polymer. The polymer of the present invention is preferably a polyester. In accordance with an embodiment of the present invention the polymer is selected from: polyesters, such as poly(ε-caprolactone) (PCL), poly(lactic acid) (PLA), poly(lactic-co-glycolic acid) (PLGA), polyglycolic acid (PGA), polyorthoesters, and polycarbonates, such as poly(trimethylene) carbonate, and copolymers thereof. Preferably, the polymer is a polyester and is preferably PCL. These polymers are often used for tissue engineering.

In accordance with an embodiment of the present invention, in the combination, the backbone of the polymer has a molar mass in the range from about 500 g·mol⁻¹ to 20000 g·mol⁻¹, preferably from 2000 g·mol⁻¹ to 10000 g·mol⁻¹. The molar mass or MM (units g/mole) of the backbone corresponds with the MM of the polymer prior to functionalization with the first thiol-ene crosslinkable group i.e. prior attachment of the first thiol-ene crosslinkable group to the polymer.

According to yet a further embodiment of the present invention, the polymer has an alkene content from 0.0001 mol/g to 0.01 mol/g, preferably from 0.005 mol/g to 0.01 mol/g. An advantage of the present embodiment is that it provides for the most beneficial crosslinking density. In particular, an alkene content higher than the one according to the present embodiment would result in too high cross-link density, which would reduce the polymer backbone length to such an extent that there is little additional value of using such a central degradable backbone, whereas an alkene content lower than the one according to the present embodiment would result in too low cross-link densities (too loose cross-linked networks), which will result in lacking mechanical integrity, improper cross-linking and inability to print via light based 3D-printing techniques.

The polymer according to the invention has a geometry selected from: linear, branched, star-shaped, preferably star-shaped. In other words, the polymer is provided with a backbone having a star-shaped backbone, a branched backbone or a linear backbone. The geometry (e.g. linear, branched and star-shaped) is determined by the initiator which is used during the polymerization of the polymer. A bifunctional initiator will lead to a linear polymer with two functional chain ends while a trifunctional initiator will lead to a branched or star-shaped polymer with three functional chain ends. According to yet a further embodiment of the present invention, the polymer is star-shaped, preferably 3-arms shaped. An advantage of the present embodiment is that the star shape, and in particular the 3-arms shape, provides for improved tunability. Having a polymer with three or more functional chain ends moreover provides for enhanced crosslinking during the thiol-ene crosslinking reaction.

The CBM comprising compound of formula (I) part of the combination according to the present invention has a group Q comprising a cell-binding motif (CBM); an optionally present spacer Z, and a group X comprising a second thiol-ene crosslinkable group.

Hence, the CBM comprising compound of formula (I) comprises a group comprising a cell-binding motif, said group denoted as Q in formula (I). By means of the term “cell-binding motif” or “CBM”, reference is made to a motif that induces specific and controlled cell adhesion. Cell adhesion is a complex combination of different sequential events: attachment, spreading, and growth which is accompanied/followed by other processes such as cell migration, differentiation, and the production of extracellular matrix (ECM) molecules. In most cases, short CBMs are peptides, yet they can also be carbohydrates, or even truly synthetic peptide-mimicking molecules (peptidomimetics). The first peptide identified from the ECM protein fibronectin was the tripeptide Arg-Gly-Asp (RGD). This tripeptide is the minimal recognition sequence for integrin receptors and is in addition to fibronectin present in other ECM proteins such as collagen, laminin, and vitronectin. RGD is sufficient and crucial for cell adhesion, it has been demonstrated that this binding motif can be modified by flanking or spatially neighbored amino acids to increase cell recognition and cell-type specificity. Plenty of different linear RGD sequence-containing peptides with varying flanking amino acid sequences have been generated to increase cell adhesion. However, it has been shown that these linear RGD derivatives can undergo slow enzymatic degradation. To circumvent this problem, cyclic RGD derivatives have been designed and conjugated to polymeric surfaces, for instance cRGDfK. Any CBM known in state of the art would be suitable to carry out the present invention. CBMs which can be used to carry out the present invention include, and are not limited to: RGD-based peptide motifs, such as RGD, RGDS, GRGD, GRGDS, GYRGDS, RGDC, RGDX (X=S, V, and T), GRGDY, GRGDSP, GRGDSPC, CGRGDSPK, GGGRGDSP, GDRGDSP, c(-RGDfK-), Acrylo-co-(RGD); peptidomimetics such as RGD-mimetics, IKVAV-based peptide/oligopeptide sequences, such as IKVAV, CIKVAV, CSRARKQAASIKVAVSADR; YIGSR-based peptide sequences such as YIGSR, CYIGSR, GYIGSR, CDPGYIGSR; and others such as KRSR, PHSRN, PDSGR, GYPDGSR; oligopeptides such as FN-C/H-V, GEFYFDLRLKGDK; carbohydrates such as galactose, lactose. According to a preferred embodiment of the present invention, the CBM comprises an RGD motif, or is RGD. An advantage of the present embodiment is that RGD makes suitable to use a variety of formulations for various tissue engineering applications like bone, cartilage, and breast implants. According to yet a further embodiment, the CBM comprising compound has the CBM being a peptide, such as RGD, and the second thiol-ene crosslinkable group being cysteine. An advantage of the present embodiment is that the compound of formula (I) can be synthesized according to peptide synthesis methodologies, thereby providing a polymer network cheaper and easier to manufacture.

The CBM comprising compound of formula (I) further comprises a spacer Z. In accordance with the present invention, by means of the term “spacer”, also known as “linker”, reference is made to a molecule, preferably a flexible molecule, used to connect at least two molecules of interest together. The spacer Z according to the present invention can be, for example, either a hydrophilic spacer, such as a PEG spacer i.e. a spacer containing one or more monomers of formula —[CH₂CH₂O]ₙ— which could be obtained from 8-amino-3,6-dioxaoctanoic acid, or a hydrophobic spacer, such as an HEX spacer i.e. a spacer containing a moiety of formula —(CH₂)₆— which could be obtained from 6-aminohexanoic acid (also referred to as Ahx). In early studies, PEG spacer and HEX spacer were selected in order to assess if a hydrophilic spacer (e.g. PEG spacer) or hydrophobic spacer (e.g. HEX spacer) would affect the cell-adhesiveness of the materials. It was surprisingly found that the hydrophilic and hydrophobic character of the linker do not influence the cell-adhesiveness as much as the length of the spacer. The spacer Z according to the present invention can hence be made from a variety of chemical compositions and structures. Conveniently, the spacer Z could be selected in such a way that the compatibility with the polymer is improved and a more homogeneous distribution (no phase separation) is achieved. Spacer can be selected to improve compatibility with the polymer to avoid phase separation.

Possible spacers Z for the present invention can be for example, a polyester spacer, such as and not limited to caprolactone, lactide, glycolide; a polyether spacer such as, and not limited to ethylene oxide, propylene oxide; aliphatic hydrocarbons such as and not limited to, methylene, ethylene, trimethylene, tetramethylene, pentamethylene, hexamethylene.

Further examples of spacer Z comprise and are not limited to: shorter/longer variants of Ahx (such as glycine, beta-alanine, aminopentanoic acid, aminoheptanoic acid, etc.), and repeats thereof, non-standard amino acids, such as including an aromatic moiety.

In accordance with the present invention, a spacer Z connects the Q group comprising a cell-binding motif with the X group, which comprises the second thiol-ene crosslinkable group. By means only optionally present, in an embodiment of the present invention the Q group and the X group are directly connected.

According to an embodiment of the present invention, the combination has Z from 0 to 30 atoms in length. The length of the spacer Z is defined as the number of atoms of the longest chain of atoms linking the group Q comprising the CBM and the group X comprising the second thiol-ene crosslinkable group. The present embodiment is advantageous in that the length of the spacer Z from 0 to 30 atoms, preferably from 0 to 10 atoms, was surprisingly found the most beneficial in providing viable cells within the polymer network obtainable from the combination of the present embodiment. With a spacer Z having a length outside the present range, a significantly reduced cell viability was observed instead. It appears that the longer the spacer, the worse the cell-adhesion properties of the polymer network of the present invention get, and that adhesion properties of the polymer network from the combination of the invention drastically decrease with a spacer of more than 30 atoms in length, and are better within 0 to 10 atoms in length. The present embodiment includes the possibility of having no spacer Z present.

According to yet a further embodiment of the present invention, the combination has Z from 1 to 30 atoms in length, preferably 1 to 10 atoms in length. The present embodiment provides all the advantages of the previous embodiment and it is further advantageous in that the spacer Z provides for a combination allowing for improved mobility of the CBM, thereby better exposing said CBM from the surface of the obtainable polymer network, whilst providing said CBM also within the bulk of the obtainable polymer network.

According to yet a further embodiment of the present invention, the combination has Z from 0 to 20 atoms in length. According to yet a further embodiment of the present invention, the combination has Z from 1 to 20 atoms in length. It was found that with the spacer Z having a length from 0 to 20 atoms, more viable cells were observed. The same trend can be seen in relation to the metabolic activity of the cells. It was found that with the spacer Z having a length from 1 to 20 atoms, more viable cells were observed, along with an increased mobility of the CBM. The same trend can be seen in relation to the metabolic activity of the cells.

According to yet a further embodiment of the present invention, in the combination, the spacer Z is of formula (II):

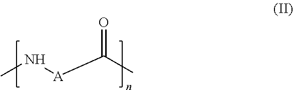

wherein:
   A is a bivalent radical, optionally comprising one or more heteroatoms; and
   n≥1, preferably 1≤n≤4.

An advantage of the present embodiment is that the structure of the spacer Z offers straightforward chemical coupling to the polymer and provides peptidic character to the spacer, which is simpler to synthesize in case Z is a peptide (e.g. by solid-phase peptide synthesis) compared to other spacers without terminal NH and C═O groups.

Group A of formula (II) is a bivalent radical obtained from the removal of a hydrogen atom from each of the two terminal carbon atoms of said group. Group A can be a variety of radicals such as alkylene groups including methylene, ethylene, methylmethylene, trimethylene, propylene, tetramethylene, ethylethylene, 1,2-dimethylethylene, pentamethylene and hexamethylene. A can also contain one or more heteroatoms, such as O or N.

The bivalent radical A is preferably from 3 to 9 atoms in length, preferably from 5 to 7 atoms in length.

According to yet a further embodiment of the present invention, n is 1. In accordance with the present embodiment the spacer Z comprises a single, non-repeating, unit. An advantage of the present embodiment is that a higher amount of adhered cells was achieved compared to n=0 or n>1.

Further, the CBM comprising compound of formula (I) comprises a second thiol-ene crosslinkable group. According to an embodiment of the present invention, the first thiol-ene crosslinkable group is a C—C double bond comprising group and the second thiol-ene crosslinkable group is a —SH comprising group. An advantage of the present embodiment is that the formation of disulfide bonds can be more easily kept at a minimum. It was found easier to control said disulfide bond formation on the linking the CBM comprising compound rather than on the polymer. By reducing the number of disulfide bonds in the combination according to the present invention, a higher crosslinking density can be achieved because more positions are available on the CBM comprising compound to crosslink with available positions on the polymer.

According to yet a further embodiment of the present invention, the second thiol-ene crosslinkable group is a C-terminal cysteine residue. An advantage of the present embodiment is that the synthesis of the CBM comprising compound can be accomplished by means of peptide synthesis in case it's a peptide (e.g. solid phase peptide synthesis (SPPS) protocol), thereby saving costs and resources. Further, cysteine is a natural standard and rather inexpensive amino acid which can easily be included during SPPS.

According to a further aspect, the present invention pertains to a polymer network comprising an outer region (at the surface of said network), an inner region i.e. bulk region, contained within the outer region, and a plurality of cell-binding motifs (CBMs), provided within the outer region and the inner region i.e, wherein CBMs are present both at the surface of said polymer network and within the bulk of said polymer network. An advantage of the polymer network according to the present aspect is that the having CBMs chemically anchored throughout the thiol-ene crosslinked polymer network significantly increased the number of adhered cells.

According to an embodiment of the present invention, the combination as defined in any one of the embodiments of the present invention in a crosslinked state. An advantage of the present aspect of the invention is that cross-linking provides additional strength and introduces great tunability over many properties, amongst which, thermal behavior, mechanical properties and degradation time.

Light-based printing offers superior resolution over deposition-based printing, nevertheless, there is a scarcity of commercially available (bio) degradable polymers that can be processed with light-based printing. By means of the composition of the present invention, it is possible to print (bio) degradable polymers by means of light-based printing. The printed networks are cell-interactive; hence useful for, among others, tissue engineering, personalized implants.

In particular, the polymer network according to the present aspect is a polymer network comprising
a polymer;
a CBM comprising compound of formula (I):

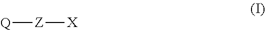

wherein:
Q is a group comprising a cell-binding motif (CBM);
Z is a spacer, optionally present;
X is a group thiol-ene crosslinked to the polymer.

By means of the term “polymer network”, reference is made to a material composed of linear strands connected by multifunctional junctions. These strands and junctions can be flexible or rigid (macro) molecules, and the connections between strands and junctions are covalent or non-covalent. The polymer networks according to the present invention comprise a thiol-ene linkage i.e. a C—S bond between the polymer and the CBM comprising compound of formula (I).

According to a further aspect, the present invention, pertains to a hydrogel comprising a polymer network in accordance with the present invention. In the context of the present invention, by means of the term “hydrogel”, reference is made to a gel wherein the swelling agent is an aqueous fluid. In other words, reference is made to a polymer network which has been expanded by means of a swelling agent. In the context of the present invention, by means of the term “swelling agent”, as used herein, unless indicated otherwise, reference is made to an agent which is capable of increasing the volume of a swellable composition according to the present invention by absorption of said agent. For example, swelling agents according to the present invention are, but not limited to, water, serum, lipo-aspirate, intravenous fluids, NaCl solution, glucose solution, Hartmann solution, stem cell solution, blood plasma, buffers, such as DMEM, HEPES, and combinations thereof.

The polymer network in accordance with the present invention can be obtained by crosslinking the combination as described in any embodiment of the present invention. The crosslinking reaction can be performed by various methods in the state of the art. The crosslinking reaction can be performed in a variety of solvents which is chosen depending on the respective solubility of both the polymer and the CBM comprising compound of formula (I).

In contrast to already reported approaches where binding motifs are introduced solely onto the surface via a so-called surface modification. Here, the binding motif is chemically linked throughout the bulk of the material during the photo-crosslinking process. Thus, as is the case in nature, the CBM is homogeneously distributed throughout the bulk of the material (e.g. gelatin). Inclusion of the CBM throughout the bulk ensures that, as the material biodegrades, novel adhesion sites will become available. Additionally, no harsh conditions, extra modification steps or degradation of the PCL is needed for the modification. To do so, cysteine-functionalized RGD was synthesized via solid phase peptide chemistry. Subsequently, the synthesized RGD-spacer-cysteine is bound throughout the network via the radical thiol-ene reaction with alkene-functionalized PCL. In addition to the introduction of the binding motif RGD, we investigated the presence (i.e. with and without), type (i.e. hydrophobic, hydrophilic) and length (i.e. 1,3 or 5 repeats) of a spacer between the RGD binding motif and cysteine coupling site. The RGD-modification is thoroughly characterized and the influence on cell adhesion of fibroblast is evaluated. It is important to note that the approach described here is robust and well-controlled. It can be hypothesized that many more biological relevant compounds can be bound throughout the PCL-bulk for several specific biological applications. The only requirement would be the presence of a cysteine (i.e. thiol). Additionally, the photo-crosslinking chemistry is not limited to PCL. It can be anticipated that many other synthetic polymers, preferably degradable polymer, which can be easily modified according to the protocol described here. Finally, due to the light-mediated photo-crosslinking process, it should be fairly easy to introduce spatial control into the system.

The polymer network according to the present invention could be used for the development of degradable implants for tissue engineering. More precisely, for the development of patient-specific-implants via light-based 3D-printing techniques.

### EXPERIMENTAL PART

### Experimental Section

Synthesis of RGD-Functionalized Peptides with Various Linkers

### Materials and Methods

N,N′-diisopropylcarbodiimide (DIC), Oxyma Pure and protected amino acid derivatives purchased from Chem-Impex, except N^{α}-Fmoc-N^{ω}-(2,2,4,6,7-were pentamethyldihydrobenzofuran-5-sulfonyl)-L-arginine (Fmoc-Arg (Pbf)-OH), and Fmoc-8-amino-3,6-dioxaoctanoic acid (Fmoc-NH-PEG-COOH) which originated from Carbosynth. Rink Amide AM resin and N,N,N′,N′-Tetramethyl-O-(1H-benzotriazol-1-yl) uronium hexafluorophosphate (HBTU) were purchased from Carbosynth, whereas trifluoroacetic acid (TFA) was obtained from Fluorochem. All other reagents and solvents used for peptide synthesis and purification originated from Merck. Analytical RP-HPLC spectra were recorded on a VWR-Hitachi Chromaster HPLC equipped with a Chromolith High Resolution RP-18C column from Merck (150 mm×4.6 mm, 1.1 μm) and a Chromaster HPLC 5430 diode array detector set at a wavelength of 214 nm. The solvent system consisted of 0.1% TFA in ultrapure water (A) and 0.1% TFA in acetonitrile (B). Analyses were performed with a linear gradient of 1% to 99% B over 4.5 min using a flow rate of 2.8 ml min-1. Peptide purifications were performed on a Gilson semi-preparative HPLC, equipped with a Supelco Discovery BioWide Pore C18 column (250 mm×21.2 mm, 10 μm) and a UV detector set at 214 nm, by using a linear gradient ranging from 1% to 40% B in 20 min using a flow rate of 20 ml min-1. LC-MS analyses were performed on a Waters 600 HPLC unit equipped with an EC 150/2 NUCLEODUR® 300-5 C18 column and a solvent system consisting of 0.1% formic acid in ultrapure water (C) and 0.1% formic acid in acetonitrile (D). A linear gradient was applied ranging from 3% to 100% D in 20 minutes at a flow rate of 0.3 ml min-1. Products were detected by a UV detector set at 214 nm, followed by MS analyses on a Micromass QTOF-micro system. HRMS was conducted on the same device with reserpine (2.1-3.0 mg ml⁻¹ in water:acetonitrile 1:1) as the reference.

### Synthetic Protocols.

Peptides were synthesized on a Rink Amide AM resin (loading 0.60 mmol/g) by standard SPPS protocols. Amino acids were used under their Na-Fmoc-protected form, combined with the following side chain protecting groups: trityl (Trt) for cysteine, tert-butyl ((Bu) for aspartic acid and 2,2,4,6,7-pentamethyldihydrobenzofuran-5-sulfonyl (Pbf) for arginine. Syntheses were carried out in polypropylene syringes equipped with a polyethylene frit using an iterative procedure consisting of Na-Fmoc deprotection and amino acid couplings. Between every step, the resin was washed thoroughly with N,N-dimethylformamide (DMF, 3 times) and dichloromethane (DCM, 3 times). Na-Fmoc deprotection steps were performed using 20% (v/v) 4-methylpiperidine in DMF for 5 and 15 minutes, respectively. Coupling of the first amino acid was performed using 3 equiv. each of Fmoc-Cys (Trt)-OH, DIC and Oxyma Pure in DMF at room temperature. All other amino acid couplings were carried out using 3 equiv. of protected amino acid, 3 equiv. of HBTU and 6 equiv. of N,N-diisopropylethylamine (DIPEA) in DMF. Completion of couplings was verified by the Kaiser colorimetric test. Peptide cleavages were performed with a freshly prepared solution of TFA/triisopropylsilane/water 90/5/5 (v/v/v) for 4 hours at room temperature. Subsequently, the resin was filtered and washed with DCM and neat TFA. The combined filtrates were evaporated, and the crude peptides were obtained after precipitation in cold diethyl ether and lyophilization. Preparative RP-HPLC purifications were performed to yield peptides with a HPLC purity exceeding 95%, as their TFA salts after lyophilization. One is referred to the supporting information for the peptides' characterization.

### Synthesis of Alkene-Functionalized PCL

### Materials

All chemicals were used as received, unless stated otherwise. ε-caprolactone (>99%), supplied by Tokyo Chemical Industry (TCI), was dried over calcium hydride (CaH₂) and vacuum distilled (120° C., 10 mbar). Allyl isocyanate (98%), glycerol (>99%), pentaerythritol (99%), Tin (II) 2-ethylhexanoate (92.5-100%), dimethyl terephthalate (99.93%) and pentaerythritol tetrakis(3-mercaptopropionate) (>95%) were supplied by Sigma-Aldrich (Diegem, Belgium). Ethyl (2,4,6-trimethylbenzoyl) phenyl phosphinate (Speedcure TPO-L, 94.5%) was supplied by Lambson Ltd HQ (West Yorkshire, UK). Toluene (>99%), chloroform (stabilised with amylene, >99%) and diethylether (stabilised with 5-7 ppm BHT, >99%) were supplied by Chem-lab NV (Zedelgem, Belgium). Toluene was distilled over sodium with benzophenone as indicator and subsequently stored on molecular sieves (4 Å). Deuterated chloroform (stabilised with silver foils+0.03% TMS, 99.8%) was supplied by Eurisotop.

### Synthesis

Poly-ε-caprolactone triol with a molar mass of 8000 g·mol⁻¹ was synthesized using the following procedure and initiator to monomer ratio of 1:69. A Schlenk equipped with a magnetic stirrer was flame-dried and ε-caprolactone (20 g, 0.175 mol, 1 eq, M=114.14 g·mol⁻¹), Sn(Oct)₂ (0.1 g, 0.247 mmol 0.5 wt % of ε-caprolactone, 405.122 g·mol⁻¹), glycerol (0.233 g, 0.0025 mol, 1:69 stoichiometric ratio initiator to monomer, 62.07 g·mol⁻¹) and anhydrous toluene (47.5 mL, 2 mol/L, 92.14 g·mol⁻¹) were added under argon atmosphere. Subsequently, three freeze-pump-thaw cycles were performed after which the reaction was left to react for 24 h at 100° C. The reaction was stopped when full conversion was verified by 1H-NMR spectroscopy. Subsequently, the obtained PCL diol was further modified to alkene-functionalized PCL (E-PCL). 1.5 eq. allyl isocyanate (1.01 g, 12.15 mmol, MM=82.09 g·mol⁻¹) was added according to the hydroxyl functionalities and the solution was left to stir for an additional 30 minutes, at 60° C. The reaction was stopped upon full conversion as verified via 1H-NMR. Ultimately, the final product was obtained as a white solid after precipitation in cold diethylether with a yield of 95%.

### Determination of Alkene-Content

For quantitative measurements, dimethyl terephthalate (DMT, 10 mg, 0.05 mmol, 1 eq., M=194.18 g·mol⁻¹) was added as internal standard to 10 mg of E-PCL 8000 and CDCl3. NMR spectra were analyzed by MestReNova software and the fully automatic baseline correction (Whittaker Smoother) was applied. The alkene content was calculated as follows:

$$Alkene content = \frac{I ( δ5 \text{.2} ) + I ( δ5 \text{.8} )}{I ( δ8 )} * \frac{N ( DMT )}{N ( alkene )} * \frac{m ( DMT )}{MM ( DMT )} * \frac{1}{m ( E - PCL )} \qquad ( 1 )$$

‘I’ stands for the integrated value of the peaks compared to the signal from DMT at 8 ppm. ‘N’ is the amount of protons of DMT and the alkene functional group. ‘m’ refers to the weighed mass and ‘MM’ refers to the molar mass.

### Photo-Curing of RGD-Functionalized Networks

First, alkene-functionalized PCL (1 g, 0.125 mmol), Pentaerythritol tetrakis(3-mercaptopropionate) (PETA-4SH, 47 mg, 0.096 mmol) and Ethyl phenyl (2,4,6-trimethylbenzoyl)phosphinate (TPO-L, 7.5 mg, 0.024 mmol) were weighed. To the mixture, 7.5.10-6 mol of the respective RGD-peptide (RGD+, PEG-RGD+, HEX-RGD+) was added as a solution in DMF (0.55 mL). The resulting mixture was then sonicated and vortexed until all components were dissolved. The latter was placed in a silicone linker, positioned between two glass plates, and irradiated with UV-A light (5 mW/cm², 30 minutes), resulting in visually homogeneous photo-crosslinked films. The light intensity was measured using the RM-12 Radiometer from Opsytec equipped with an UVA sensor. The irradiation set-up was equipped with 4 light-bulbs (350 blacklight Sylvania).

### Characterization

¹H-NMR spectroscopy was performed using a Bruker Avance 400 MHz NMR Spectrometer. 16 scans were recorded with a relaxation delay of 1 second and a spectral width of 20 ppm. For quantitative 1H-NMR using DMT as internal standard, the relaxation delay was set at 10 seconds. Thermogravimetric analyses were performed starting from 30° C. up to 600° C. at a heating rate of 10° C./min. The degradation temperature was determined as the 2 wt. % mass loss. A sample mass of 10 mg was used. The analyses were performed on a TGA Q50 (TA instruments). Samples were measured in a platinum pan under a nitrogen flow of 60 ml/min. A nitrogen flow of 40 ml/min was used for stabilizing the balance. The glass transition temperature, crystallization temperature and melting temperature were determined using differential scanning calorimetry (DSC) at heating scans from −80° C. to 100° C. Multiple heating and cooling cycles were performed, according to the following method: 1. equilibrate at 20° C.; 2. First heating cycle at a heating rate of 10° C./min to 100° C.; 3. First cooling cycle at a cooling rate of 10° C./min to −80° C.; 4. Second heating cycle at a heating rate of 10° C./min to 100° C. DSC measurements were performed using a DSC Q2000 (TA Instruments), RSC 500 cooler (Zellik, Belgium) and 5 mg of sample. Each sample was measured in an aluminum Tzero pan under nitrogen flow. An empty pan was used as reference. The Q series software was used to analyze the DSC thermograms. Gel fraction and swelling properties were determined on discs (4 mm diameter) which were punched out of 2D crosslinked films and immersed during four days in an excess of chloroform. The mass of the discs (3 mm diameter) was measured when swollen (Ws) and in dry state after swelling (Wf). Using equations (2) and (3), the gel fraction and swelling ratio were calculated, respectively. All measurements were performed in triplicate.

$$Gel fraction ( \% ) = \frac{Wf}{Wd} \times 100 \qquad ( 2 )$$

$$Swelling ratio ( \% ) = \frac{Ws - {W}_{f}}{{W}_{f}} \times 100 \qquad ( 3 )$$

Static contact angle measurements (SCA) were performed using an OCA20 apparatus in sessile mode from DataPhysics Instruments Gmbh. To study the surface chemical composition, X-ray photoelectron spectroscopy (XPS) was performed using a S-Probe Monochromatized XPS spectrometer from Surface Science Instruments (VG). Source: AlKα x-ray (1486.6 eV) monochromatic, take off angle: q=45°; Voltage and power of the source 10 kV, 200 W. A flood gun and Ni grid were used for compensation of charging effects. The XPS survey spectra were collected with a pass energy Ep=140.83 eV and energy steps Es=0.160 eV. The base pressure of the XPS chamber was 1×10−7 Pa. All XPS spectra were analyzed using the CasaXPS.

### Biological Evaluation.

Dulbecco's Modified Eagle Medium (DMEM, Sigma-Aldrich, BE) supplemented with 10% (v/v) Foetal Bovine Serum (FBS, Sigma-Aldrich, BE) and 1% (v/v) penicillin/streptomycin (Sigma-Aldrich, BE) was used to culture Human Foreskin Fibroblasts (HFF, ATCC) at 37° C. in 5% CO₂. Every three days, the culture medium was changed until reaching 80-90% confluency which was followed by sub-culturing. The photo-cured samples for cell culture were incubated in a 70% (v/v) ethanol solution for two cycles of 12 hours as a first sterilization step. Thereafter, UV-C irradiation (100-280 nm, 15 mW/cm²) was applied. To observe the cell-biomaterial interaction, 10 000 HFF/cm² at passage number 11 were seeded on top of the sterile photo-cured samples in a 96 well plate. After one, three and seven days, the viability and morphology was investigated through a Live/Dead (Calcein-acetoxymethyl (CA-AM, Sigma-Aldrich, BE)/Propidium iodide (PI, Sigma-Aldrich, BE)) staining. Moreover, after one day of culture, the metabolic activity of the seeded cells was quantified through a 3-(4,5-dimethylthiazol-2-yl)-5-(3-carboxymethoxyphenyl)-2-(4-sulfophenyl)-2H-tetrazolium (MTS, Abcam, NE) assay. Regarding the Live/Dead staining, a 2% (v/v) CA-AM/PI in phosphate buffered saline (PBS) solution was applied to the seeded cells which was followed by a dark incubation at room temperature for 10 minutes. A fluorescence microscope (Olympus IX81 with Xcellence Pro software) equipped with a green fluorescent protein (GFP) and a Texas Red (TxRed) filter was used to visualize the seeded cells. FIJI software was used to compute the percentage viability. Furthermore, the seeded cells were supplemented with 17% (v/v) MTS in culture medium solution in order to quantify the cell metabolic activity. After incubation in the dark at 37° C. for 2 hours under continuous shaking, the absorbance was quantified at 490 nm with a spectrophotometer (BioTek Instruments, EL800 Universal Microplate Reader, with GEN5 software).

### Results and Discussion

Synthesis and Characterization of RGD-Functionalized Peptides with Various Linkers

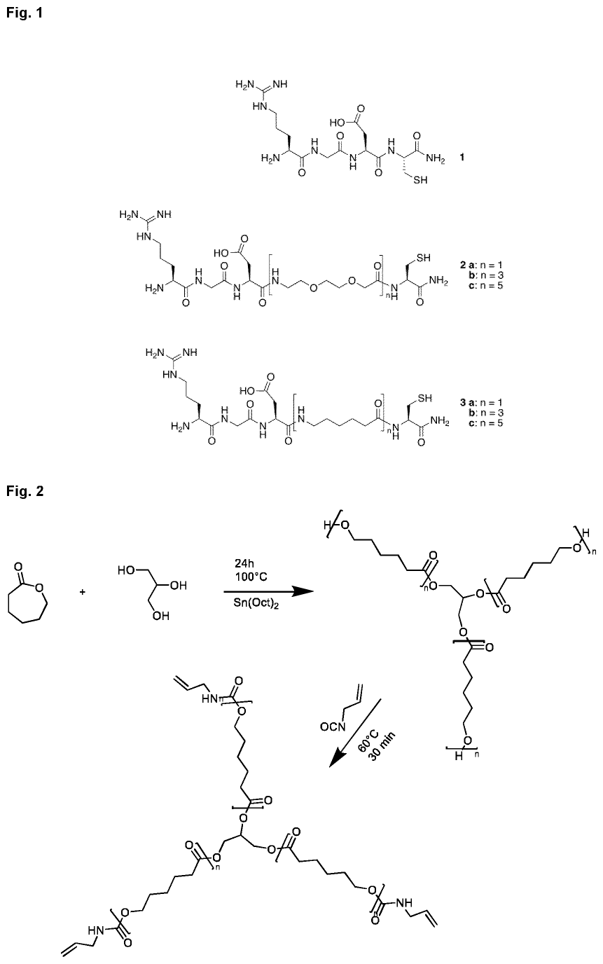

To enable linkage of the cell adhesive RGD motif to alkene-functionalized PCL, short peptide sequences were considered wherein the RGD sequence was linked to an additional N-terminal cysteine residue (see FIG. 1). This linkage was either performed directly (1), or through introduction of the spacing amino acids 8-amino-3,6-dioxaoctanoic acid (PEG, 2a) or 6-aminohexanoic acid (HEX, 3a). Whereas the HEX linker involves a standard hydrophobic alkane chain, the PEG linker introduces a higher hydrophilicity. In addition, repeats of these linking amino acids were introduced to elongate the linker lengths in sequences 2b-c and 3b-c.

All peptide sequences were synthesized as C-terminal amides by application of standard Fmoc/tBu-based solid-phase peptide synthesis (SPPS) conditions on a Rink amide-modified aminomethylpolystyrene resin. To suppress racemization during coupling of the C-terminal cysteine residue, this residue was activated using diisopropylcarbodiimide (DIC) and Oxyma Pure, whereas all other residues were activated by the aminium salt HBTU. All peptides were obtained under their TFA salt form after preparative HPLC purification, with a (HPLC) purity exceeding 95%.

### Synthesis and Characterization of Alkene-Functionalized PCL

Alkene-functionalized PCL was synthesized according to a two-step one-pot reaction scheme (see FIG. 2, Table 1). First, star-shaped PCL diol was polymerized via metathesis ring opening polymerization using tin (octanoate) as catalyst and glycerol as initiator. An initiator to monomer ratio of 1:69 was used to target a molar mass of 8 000 g·mol⁻¹. A conversion of 99% was confirmed by 1H-NMR after 24 hours of reacting. Subsequently, the terminal hydroxyl functionalities were employed to introduce alkenes by means of a post polymerization modification (PPM). To do so, allyl isocyanate was introduced and quantitative conversion was verified by 1H-NMR within 30 minutes. Notably, the catalyst used for the polymerization of PCL, tin octanoate, also is an efficient catalyst for the alcoholysis of isocyanates. Moreover, the PPM was confirmed by 1H-NMR as the protons corresponding to the end-standing CH₂-moieties shifted from 3.6 to 3.7 ppm. By means of DMT as internal 1H-NMR standard, an alkene content of 3.78·10⁻⁴ mol·g⁻¹ was determined. Furthermore, an effective molar mass of 8032 g·mol⁻¹ was obtained via 1H-NMR, illustrating excellent agreement with the intended molar mass. Based on this molar mass and the determined alkene content, a degree of substitution (DS) of 101% was determined (Supporting information). Clearly, the actual DS is more likely to be 100% and the 1% difference can be explained by a slight deviation from the actual molar mass. Indeed, when determining relatively high molar masses (i.e., 8000 g·mol⁻¹) by means of ¹H-NMR, it is reasonable to expect a 1% deviation. Overall, it can be concluded that alkene-functionalized PCL was synthesized in an extremely controlled manner leading to a DS that approaches 100%.

TABLE 1
Chemical characterization of E-PCL 8000.

| | | | | | | Degree |
|---|---|---|---|---|---|---|
| | Target | | | Con- | Alkene | of sub- |
| | molar | Molar mass | Yield | version | content | stitution |
| | mass | (¹H-NMR) | (%) | (%) | (mmol · g⁻¹) | (%) |
| E-PCL | 8000 | 8032 | 95 | 99 | 3.78 · 10⁻⁴ | 101 |
| 8000 | | | | | | |

### RGD-Functionalized PCL Via Thiol-Ene Cross-Linking

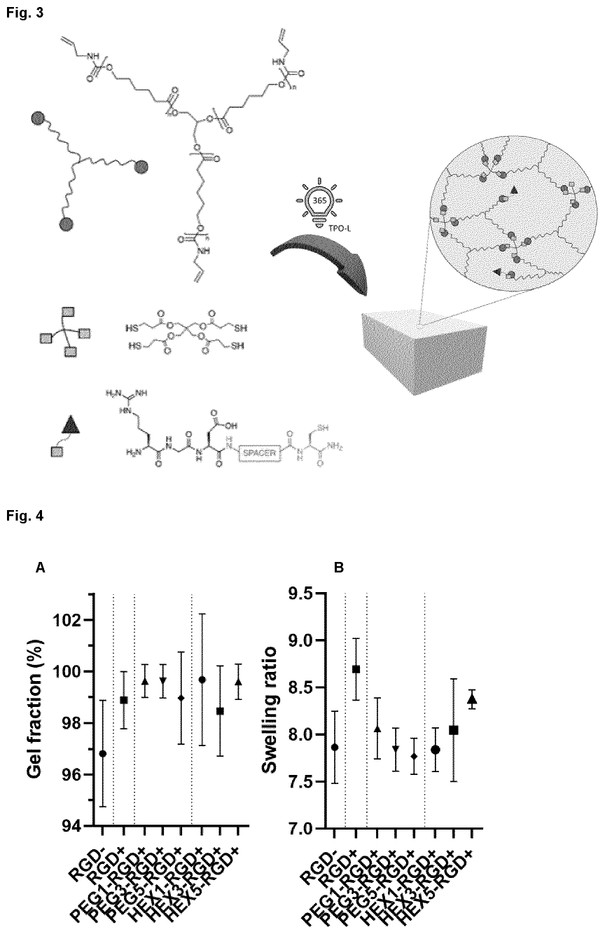

Next, the RGD-peptides were chemically bound throughout the thiol-ene cross-linked PCL-networks (see FIG. 3). To do so, the respective peptides were introduced into the curing mixture as they were photo-cross-linked. More precisely, eight different networks were prepared. Firstly, a benchmark PCL-network was prepared, without RGD (i.e., RGD-). Secondly, a PCL-network was functionalized with RGD but without a linker between the CBM and cysteine coupling site (cfr. 1 in FIG. 1; i.e., RGD+). Next, three PCL-networks were functionalized with RGD were the CBM was linked to the cysteine coupling site by 1, 3 or 5 repeats of the hydrophilic linker (cfr. 2a-c in FIG. 1; i.e., PEG1-RGD+, PEG3-RGD+ and PEG5-RGD+). Finally, three PCL-networks were functionalized with RGD, where the CBM was linked to the cysteine coupling site by 1, 3 or 5 repeats of the hydrophobic linker (cfr. 3a-c in FIG. 1; i.e., HEX1-RGD+, HEX3-RGD+ and HEX5-RGD+). We opted for the described experimental design based on the following rational. Firstly, it was attempted to assess the influence of the presence and length of a linker between the cell-binding motif (i.e., RGD) and the cysteine coupling-site on the cell adhesiveness of the PCL-networks. Secondly, it was attempted to assess the influence of the nature (i.e., hydrophilic/hydrophobic) of the linker on the cell adhesiveness of the PCL-networks.

### Characterization of the RGD-Functionalized Thiol-Ene Cross-Linked PCL-Networks

First, to identify any influence of the presence of the peptides on the materials' physicochemical properties, the materials were thoroughly characterized. The network connectivity of the materials was evaluated through gel fraction and swelling experiments. Gel fractions ranged between 96 and 100%, indicating excellent network connectivity (FIG. 4A). Furthermore, swelling degrees ranged between 7.5 and 9 for all materials without any significant differences between the different materials (FIG. 4B). Moreover, it was shown by TGA that negligible degradation occurred prior to 200° C. illustrating excellent thermal properties, and again, no differences between the different samples could be identified. Additionally, this confirms that DMF (used during cross-linking) was effectively removed from the cross-linked PCL-networks. Finally, by means of differential scanning calorimetry (DSC), it was shown that the peptides' presence did not affect the melting/crystallization behavior of the networks, as the melting temperature ranged between 41-42° C. and the melting enthalpies ranged between 42-45 J/g for all materials. Interestingly, these result show that incorporation of the peptide into the PCL-networks did not affect their physicochemical properties.

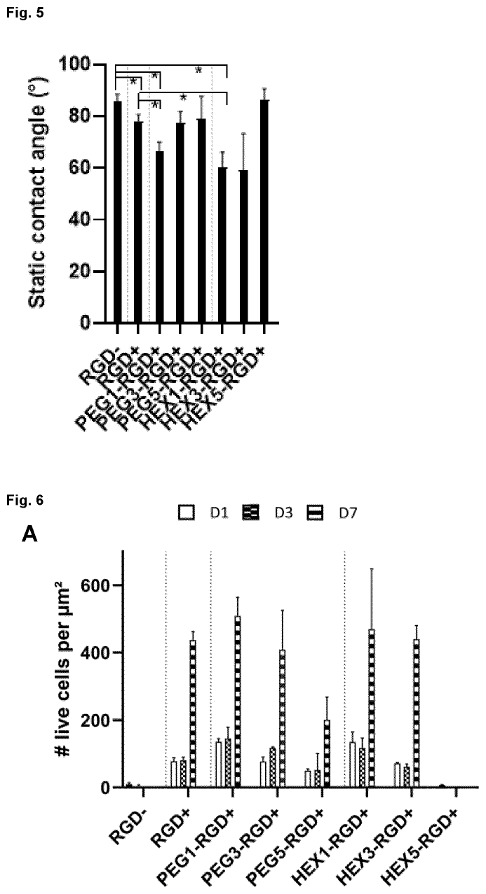

Illustrating the modification. To get insight into whether the RGD-peptides are effectively incorporated into the networks, static contact angle (SCA) measurements were performed (FIG. 5). It was hypothesized that the presence of RGD-containing peptides would increase the hydrophilicity of the materials. Indeed, a significant (P<0.05) reduction of the static contact angle (SCA) was seen upon introduction of RGD (i.e., RGD− versus RGD+). Interestingly, introduction of a small linker (n=1), between the CBM and the cysteine coupling site, further reduced the SCA, as compared to the RGD-functionalized network without linker (i.e., RGD+ versus PEG1-RGD+/HEX1-RGD+). Since peptides can be considered rather hydrophilic, it can be assumed that they would be more inclined to reside at the surface of the network. Therefore, it is hypothesized that this migration towards the surface is more pronounced due to the increased mobility of the peptide, thereby further increasing the SCA. However, when comparing longer linker sizes (n=3 or 5) with the non-functionalized PCL-network, no significant differences were observed. Nevertheless, in case of n=3, a reduction of the SCA could still be observed compared to the non-functionalized PCL-network, although not significant. These results indicate that the largest difference in hydrophilicity of the PCL's surface resulted from the introduction of RGD-peptides that contained a short linker (n=1). However, caution is advised when attributing this absence of SCA reduction to the presence of increased linker size. Indeed, it is also possible that the increased linker size renders the peptide less reactive, thereby resulting in the inability to be properly incorporated into the network.

In order to investigate the presence of RGD on the surface more quantitatively, X-ray photoelectron spectroscopy (XPS) was performed (see Table 2). XPS can provide information on the atomic abundancy in the most upper layer of the surface (i.e., analysis depth of 5 nm). For the unmodified material (i.e., RGD-), only limited presence of nitrogen was found (0.21 wt. %). This atomic abundancy of nitrogen stems from the carbamate functionalities that are present within the PCL-network (resulting from the reaction between the isocyanate and the alcohol). Introduction of RGD without linker increased the presence of nitrogen at the surface to 1.35 wt. %. Furthermore, introduction of RGD having 1, 3 and 5 repeats of the hydrophilic linker (PEG) led to atomic abundancies of 3.54, 3.05 and 0.78 wt. %, respectively. Finally, introduction of RGD having 1, 3 and 5 repeats of the hydrophobic linker (HEX) led to atomic abundancies of 2.97, 0.46 and 0.39 wt. %, respectively. These results correspond well with the previously described SCA (vide supra). Introduction of RGD led to an increased presence of nitrogen at the surface for all PCL-networks, as compared to the unmodified PCL-network. Interestingly, also here it was found that a short linker increased the presence of nitrogen at the materials' surface considerably compared to the RGD-functionalized PCL-network without linker. Finally, as was seen in SCA, the increased presence of nitrogen is again less pronounced for longer linker lengths, and considerably smaller for the longest linker (n=5). However, it should be noted that even for the longest linker, the atomic abundancy of nitrogen was still at least 2-fold increased, as compared to the non-functionalized PCL-network. These results indicate that there is no clear impact of using a hydrophilic (i.e., PEG) or hydrophobic (i.e., HEX) linker. However, the use of moderate linker lengths (n=1) led to a 15-fold increase in atomic abundancy of nitrogen thereby clearly illustrating the presence of RGD at the networks' surfaces. Notably, the low amounts of Fluor, Silicon and Tin that were measured stem from to the polytetrafluoroethylene (PTFE) non-adhesive foil, silicone spacer that was used during photo-crosslinking of the materials and the catalyst that was used for the PCL synthesis (i.e., tin (octanoate)).

TABLE 2
Atomic abundancy (in wt. %) at the surface of the RGD-functionalized
PCLs determined via XPS.

| | C 1s [%] | F 1s [%] | N 1s [%] | O 1s [%] | Si 2p [%] | Sn 3d [%] | Total [%] |
|---|---|---|---|---|---|---|---|
| RGD− | 68.64 | | 0.21 | 24.82 | 6.33 | | 100 |
| RGD+ | 67.48 | | 1.35 | 24.00 | 7.17 | | 100 |
| PEG1−RGD+ | 66.39 | 1.32 | 2.97 | 23.21 | 6.00 | 0.14 | 100 |
| PEG3−RGD+ | 68.02 | | 0.46 | 26.99 | 4.52 | 0.03 | 100 |
| PEG5−RGD+ | 67.54 | 1.44 | 0.39 | 26.45 | 5.14 | | 101 |
| HEX1−RGD+ | 66.24 | 1.42 | 3.54 | 23.14 | 5.50 | 0.16 | 100 |
| HEX3−RGD+ | 67.32 | 0.90 | 3.05 | 24.24 | 4.38 | 0.11 | 100 |
| HEX5−RGD+ | 67.54 | | 0.78 | 23.10 | 8.58 | | 100 |

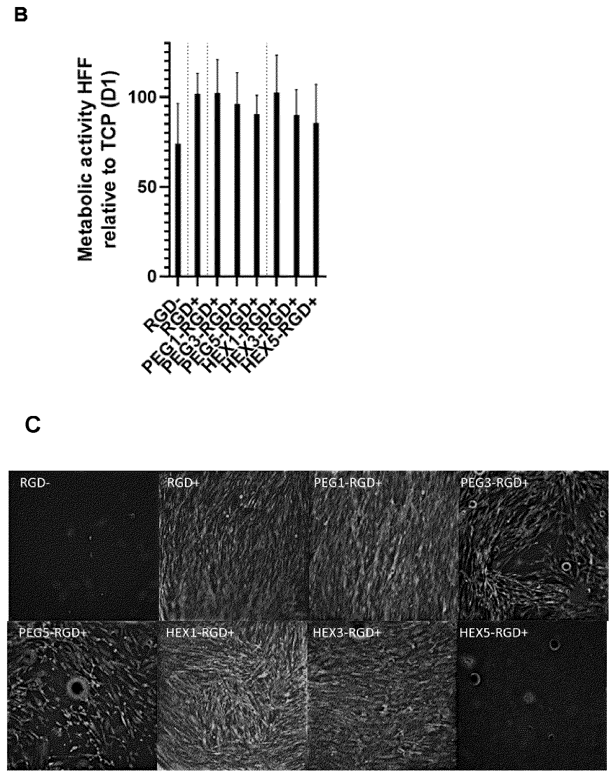

Evaluation of cell adhesive properties. The presence of RGD on the surface of the thiol-ene cross-linked PCL networks was illustrated via SCA and XPS (vide supra). In order to evaluate the effect of the presence of RGD on cellular behavior (i.e., adhesion and proliferation), human foreskin fibroblasts (HFFs) were cultured for 1, 3 and 7 days on the different RGD-functionalized PCLs after which the cell adhesion was assessed via a life/death assay. The presence of RGD had a clear effect, corresponding well with the above findings (i.e., SCA/XPS). However, in order to quantify this effect, the number of alive cells on the materials' surfaces were counted and normalized according to the materials' surface (i.e., number of cells per square μm) (FIG. 6A) Shortly after initial seeding (1 day), almost no cells adhered onto the non-functionalized PCL-network. For RGD without linker, the number of adhered cells was 78±10 per μm². Furthermore, introduction of RGD with 1, 3 and 5 repeats of the hydrophilic linker (PEG) led, respectively, to 135±9, 77±13 and 50±5 adhered cells per μm². Finally, introduction of RGD with 1, 3 and 5 repeats of the hydrophobic linker (HEX) led, respectively, to 135±30, 72±3 and 7.5±1 adhered cells per μm². These results correspond well with the presence of RGD on the networks' surfaces (vide supra). Clearly, chemically anchoring of RGD throughout the thiol-ene photo-cross-linked PCL-networks significantly increased the number of initially adhered cells. Furthermore, the number of initially adhered cells could be increased 2-fold by introducing a short linker (n=1), where the nature of the linker (i.e., hydrophilic or hydrophobic) did not seem to affect the cell adhesiveness significantly. Although, in case of n=5, the hydrophilic linker performed better as compared to the hydrophobic linker. Indeed, despite the low initial cell adhesiveness, a confluent monolayer of HFFs was still present after 7 days (FIG. 6C). The effect of increased linker length was less pronounced for n=3 and not present anymore in case of n=5. Furthermore, the effect of the RGD-functionalization on the cells' metabolic activity was evaluated by means of an MTS assay (FIG. 6B). These results correspond well with live/dead assay, as cells on unmodified PCL were considerably less metabolically active. Finally, culturing of HFFs for a longer time (i.e., 3 and 7 days) showed viable cells that were able to reach confluency on all materials for which sufficient initial cell adhesion was present (FIG. 6C).

Volumetric 3D-printing of cell-adhesive scaffolds. This example illustrates the use of the described invention for creating cell-adhesive scaffolds through volumetric 3D-printing in a single step. Volumetric 3D-printing, a light-based technique, enables manufacturing of complex designs in seconds by using light to spatiotemporally initiate photo-crosslinking of liquid resin into the desired 3D geometry. A specialized resin for volumetric 3D-printing was designed, comprising a star-shaped alkene-functionalized PCL oligomer, a tetra-functional thiol crosslinker, DMF as a solvent, TPO as the photo-initiator, and H-Arg-Gly-Asp-E-Ahx-Cys-NH₂ as the cell-binding motif.

Using this composition, 3D constructs were volumetrically printed with a commercially available volumetric 3D printer (Tomolite, sold by Readily3D), loaded with 0, 0.1, and 0.2 wt. % of the cell-binding motif.

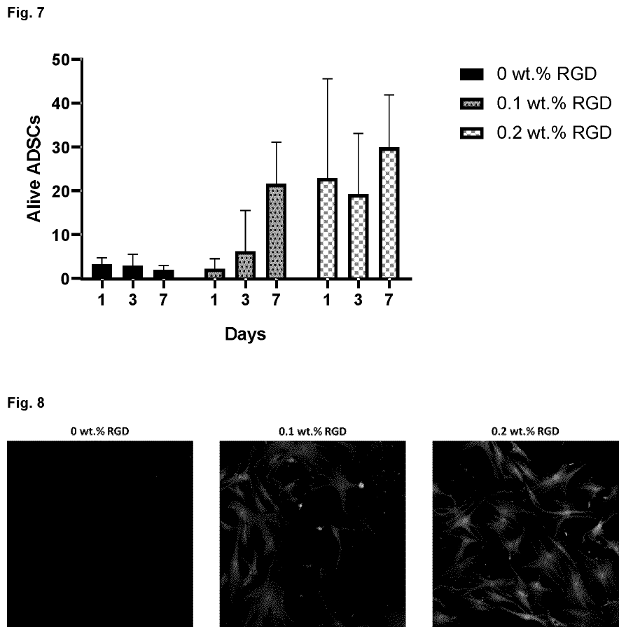

The cell-adhesiveness of these constructs was evaluated by seeding adipose-derived stem cells (ADSCs) and determining the quantity of living cells adhered to the material surfaces after 1, 3, and 7 days. The results of quantification of living adipose derived stem cells (ADSCs), after 1, 3 and 7 days, on the surface of 3D photo-crosslinked polyester scaffolds, obtained by volumetric 3D-printing of a photoresist that contained RGD as cell-binding motif, is represented in FIG. 7. A representation of visualization of living adipose derived stem cells (ADSCs), after 7 days, on the surface of 3D photo-crosslinked polyester scaffolds, obtained by volumetric 3D-printing of a photoresist that contained RGD as cell-binding motif, is provided in FIG. 8.

The quantification of living ADSCs illustrates that, upon introducing the cell-binding motif into the non-hydrogel-based resin, cell-adhesiveness could be achieved on the surface of the scaffolds. This observed enhancement of cell-adhesiveness confirms the unexpected finding related to the present invention, where integrating a cell-binding motif homogeneously throughout the bulk of a material results in cell-adhesiveness at the surface of the resulting photo-cured part. It should be noted that the current invention involves non-hydrogel materials, which do not swell in water, leading to an expectation that the peptide would have limited mobility. Surprisingly, although the peptide was incorporated starting from a homogeneous photoresist, the incorporated peptides are present at the surface and have sufficient mobility to enable cell-attachment.

### Abbreviations

ECM=extra cellular matrix, CBM=cell-binding-motif, PCL=poly(ε-caprolactone), RGD=Arg-Gly-Asp, EDC=, NHS, DIC=N,N-diisopropylcarbodiimide, Fmoc-Arg (Pbf)-OH═N^{α}-Fmoc-N^{ω}-(2,2,4,6,7-pentamethyldihydrobenzofuran-5-sulfonyl)-L-arginine, Fmoc-NH-PEG-COOH=Fmoc-8-amino-3,6-dioxaoctanoic acid, HBTU=N,N,N′,N′-Tetramethyl-O-(1H-benzotriazol-1-yl) uronium hexafluorophosphate, TFA=trifluoro acetic acid, Trt=trityl, tBu=tert-butyl, Pbf=2,2,4,6,7-pentamethyldihydrobenzofuran-5-sulfonyl, DMF=dimethyl formamide, DIPEA=N, N-diisopropylethylamine, DCM=dichloromethane, DMT=dimethyl terephthalate, TPO-L=Ethyl phenyl(2,4,6-trimethylbenzoyl)phosphinate, MM=molar mass, PETA-4SH=Pentaerythritol tetrakis(3-mercaptopropionate), TGA=thermogravimetric analysis, DSC=differential scanning calorimetry, XPS=X-ray photoelectron spectroscopy, DMEM=Dulbecco's Modified Eagle Medium, FBS=Foetal Bovine Serum, HFF=human foreskin fibroblasts, Ca-AM=Calcein-acetoxymethyl, MTS=3-(4,5-dimethylthiazol-2-yl)-5-(3-carboxymethoxyphenyl)-2-(4-sulfophenyl)-2H-tetrazolium, PBS=phosphate buffered saline, DIC=N,N′-diisopropylcarbodiimide, PTFE=polytetrafluoroethylene, PEG=polyethylene glycol, HEX=6-aminohexanoic acid.

### REFERENCES

1. Causa, Filippo, et al. “Surface investigation on biomimetic materials to control cell adhesion: the case of RGD conjugation on PCL.” Langmuir 26.12 (2010): 9875-9884.
2. Zhang, Huina, and Scott Hollister. “Comparison of bone marrow stromal cell behaviors on poly(caprolactone) with or without surface modification: studies on cell adhesion, survival and proliferation.” Journal of Biomaterials Science, Polymer Edition 20.14 (2009): 1975-1993.

1. A combination comprising
a polymer, preferably a degradable synthetic polymer, having a first thiol-ene crosslinkable group;
a cell-binding motif (CBM) comprising compound of formula (I): 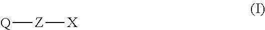
wherein:
Q is a group comprising a CBM;
Z is a spacer, optionally present;
X is a group comprising a second thiol-ene crosslinkable group;
wherein at least one of said first and second thiol-ene crosslinkable group is a thiol comprising group and at least one of said first and second thiol-ene crosslinkable group is a -ene comprising group;
wherein the polymer is selected from: polyesters, polyorthoesters, polycarbonates, and copolymers thereof; and
wherein the polymer is star-shaped.

2. The combination according to the previous claim, wherein the polymer is a polyester selected from: poly(ε-caprolactone) (PCL), poly(lactic acid) (PLA), poly(lactic-co-glycolic acid) (PLGA), and polyglycolic acid (PGA).

3. The combination according to any one of the previous claims, wherein the spacer Z is from 0 to 30 atoms in length.

4. The combination according to any one of the previous claims, wherein the spacer Z is from 0 to 10 atoms in length.

5. The combination according to any one of the previous claims, wherein the spacer Z is from 1 to 30 atoms in length, preferably from 1 to 10 atoms in length.

6. The combination according to any one of the previous claims, wherein the spacer Z is of formula (II): 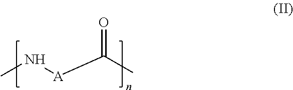
wherein:
A is a bivalent radical, optionally comprising one or more heteroatoms; and
n≥1, preferably 1≤n≤4.

7. The combination according to the previous claim, wherein n is 1.

8. The combination according to any one of the previous claims where the CBM comprises an RGD motif.

9. The combination according to any one of the previous claims, wherein the polymer is 3-arms shaped.

10. The combination according to any one of the previous claims, wherein the first thiol-ene crosslinkable group is a C—C double bond comprising group and the second thiol-ene crosslinkable group is a —SH comprising group.

11. The combination according to the previous claim, wherein the second thiol-ene crosslinkable group is a C-terminal cysteine residue.

12. The combination according to any one of the previous claims wherein the polymer has an alkene content from 0.0001 mol/g to 0.01 mol/g, preferably from 0.005 mol/g to 0.01 mol/g

13. The combination according to any one of the previous claims, wherein the polymer has a molar mass from 500 g/mol to 20000 g/mol.

14. A polymer network comprising:
an outer region;
an inner region; and
a plurality of cell-binding motifs (CBMs), provided within the outer region and the inner region.

15. The polymer network according to claim 14, comprising the combination as defined in any one of claims 1 to 13 in a crosslinked state.
